(19) United States
(12) Patent Application Publication
(10) Pub. No.: US 2004/0222108 A1
(43) Pub. Date: Nov. 11, 2004

(54) Method and system for delivering a fragrance to medical apparel packaged in a container

(76) Inventors: Domenic Tommarello, Mars, PA (US); John R. Berkley, Bridgewater, CT (US)

Correspondence Address: David Prashker, DAVID PRASHKER, P.C., P.O. Box 5387, Magnolia, MA, 01930, US

(21) Appl. No.: 10/806,530

(22) Filed: Mar. 23, 2004

Related U.S. Application Data
(60) Provisional application No. 60/459,949, filed on Apr. 3, 2003.

(51) Int. Cl. A61M 16/00; A62B 18/02

(52) U.S. Cl. 206/205; 206/438

(57) ABSTRACT

The present invention provides an article of manufacture, a sealed container of fabric medical articles scented in-situ; and also offers a method and system for scenting in-situ a sealed container of previously constructed fabric articles. This method and system of scenting the raw fibrous materials is indirect; and the structural fabric components comprising a non-woven, woven, or other fabric medical item do not change, and are not added to or subtracted from its intended barrier performance. Also, this method and system of delivering a fragrance of choice to a previously manufactured fabric article does not alter any material, does not modify any structure, and does not affect any physical or chemical property and feature of the fabric and medical article itself.

### PRIORITY CLAIM

The instant invention was first filed as U.S. Provisional Patent Application Ser. No. 60/459,949 on Apr. 3^{rd}, 2003.

### FIELD OF THE INVENTION

The present invention is concerned generally with improvements in the manufacture of scented medical apparel and other medical articles; and is particularly directed to a system for delivering and impregnating a fragrance to fully constructed medical articles, such as facemasks, which have been previously collected and packaged within a sealed dispenser.

### BACKGROUND OF THE INVENTION

Federal law requires health care providers to wear infection control apparel such as surgical facemasks. Surgical facemasks and similar fabricated non-woven devices generally are assembled from a variety of material layers each comprised of fibrous materials that are supplied by various mills. These surgical facemasks are porous in nature; and are designed to trap sub-micron particles and bacteria at very high efficiencies, typically equal to or greater than 99% @ 0.1 micron in barrier form.

Various materials are employed in fabricating a facemask. The outer-facing of a face mask is usually formed as a pulp/polyester or polypropylene spunbond; the filter of a facemask typically is formed of polypropylene meltblown; the inner-facing of a facemask is primarily formed of pulp/polyester; and the binding tapes of the face mask are primarily formed of polypropylene or polyester. Unfortunately, all of these materials are very effective at trapping and holding odors. Consequently, when in use and because the facemask fits closely to the nose, any previously captured odor may be uncomfortable or even offensive to an odor-sensitive wearer.

The Intrinsic Odor of a Facemask:

Every fibrous material employed in the making of a fabricated non-woven article such as a face mask has some intrinsic level of odor that is the combined result of the raw material fiber, the manufacturing process, the packaging and the assembly plant. Combining different fibrous materials provided by a variety of suppliers also changes the odor and intensity of the facemask. In addition, the same raw material provided by a single supplier can have a higher odor level from order to order and batch to batch. There is no known way to predict what odor will be detectable or how intense the odor will be for any supplied material.

Generally, assuming a low level of odor, a caregiver (physician, surgeon, nurse and the like) becomes familiar with a particular odor emanating from a facemask over a period of two (2) weeks or less. After this familiarizing period, the wearer no longer notices a particular odor. An odor-sensitive wearer, however, will notice subtle differences from facemask to facemask and aggressively resist changing facemask types and manufacturers. There is no familiarizing period for this type of wearer. Also, the power of suggestion is so strong in some wearers of facemasks that the individual will develop a rash, facial discoloring or other visible clinical manifestations in response to the odor emanating from the facemask.

The Scope of the Problem:

Facemasks that have a predictable pleasant scent and odor intensity significantly reduce a wearer's difficulty in changing manufacturers, changing facemask types or wearing a facemask at all. Also, it is estimated that odor-sensitive wearers consist of about 3% of the total users of facemasks. On this basis, it is presumed that 15% of non-odor-sensitive wearers would insist on a scented facemask and 85% of odor-sensitive wearers would insist on a scented facemask.

Preliminary evaluations of spearmint scented facemasks by nurses indicate that everyone likes a pleasant scent. Odor-sensitive caregivers indicated that they could easily familiarize with and comfortably wear a spearmint-scented facemask for long durations of time. It will be noted also that there are an estimated 3,000,000 cases of facemasks per year (900,000,000 facemasks annually) sold to medical facilities in North America alone.

Using these premises as a basis therefore yields a conclusion that 500,000 cases (or 150,000,000 individual facemasks) are typically used annually. Clearly, the commercial need and overall aesthetic value for a specifically scented and odor pleasant facemask (and similarly fabricated textile articles) thus are well established.

Nevertheless, in so far as is known to date, no practical technique has yet been created which would allow fully constructed medical articles, such as face masks, to become scented after their manufacture in such as manner that they would effectively become impregnated with and subsequently retain a pre-chosen fragrance, especially after the constructed articles have been packaged in particular quantities and sealed within a dispenser.

### SUMMARY OF THE INVENTION

The present invention has multiple aspects. A first aspect is a sealed container of constructed medical articles scented in-situ comprising:

a closed dispenser housing comprising at least one wall of predetermined external dimensions and configuration and having a closed internal volume of air;

a sealed opening in said wall of said dispenser housing which can be unsealed at will for the on-demand dispensation of a constructed medical article scented in-situ;

an air permeable barrier of determinable dimensions and configuration positioned within said closed internal volume of said dispenser housing such that at least first and second isolated spatial pockets of air are formed;

a predetermined number of constructed medical articles positioned within said first isolated spatial air pocket;

a fixed quantity of at least one scent-releasing fragrant material positioned within said second isolated spatial air pocket, wherein the scent of said fragrant material is released into and becomes carried by the air of said second isolated spatial pocket;

a migratory scented air mass generated within said second isolated spatial pocket which passes through said air permeable barrier and intermixes with the air of said first isolated spatial pocket, whereby said constructed medical articles become exposed to said scented air mass and become impregnated with said scent in-situ while contained within said first isolated spatial pocket of said closed dispenser housing.

A second aspect of the instant invention is a method for making a sealed container of constructed medical articles scented in-situ, said method comprising the steps of:

obtaining a closed dispenser housing comprising at least one wall of predetermined external dimensions and configuration and having a closed internal volume of air;

introducing a sealed opening in said wall of said dispenser housing which can be unsealed at will for the on-demand dispensation of a constructed medical article scented in-situ;

positioning an air permeable barrier of determinable dimensions and configuration within said closed internal volume of said dispenser housing such that at least first and second isolated spatial pockets of air are formed;

placing a predetermined number of constructed medical articles within said first isolated spatial air pocket;

putting a fixed quantity of at least one scent-releasing fragrant material within said second isolated spatial air pocket, wherein the scent of said fragrant material is released into and becomes carried by the air of said second isolated spatial pocket as a scented air mass;

allowing said scented air mass generated within said second isolated spatial pocket to pass through said air permeable barrier and intermix with the air of said first isolated spatial pocket, whereby said constructed medical articles become exposed to said scented air mass and become impregnated with said scent in-situ while contained within said first isolated spatial pocket of said closed dispenser housing.

### BRIEF DESCRIPTION OF THE DRAWING

The present invention may better appreciated and more easily understood when taken in conjunction with the accompanying Drawing, in which:

FIG. 1 shows an exploded view of the requisite elements and component parts comprising a scented dispenser of facemasks; and

FIG. 2 shows the elements and components of a scented dispenser of facemask prior to their assembly as a sealed package.

### DETAILED DESCRIPTION OF THE INVENTION

The present invention is an article of manufacture, a sealed container of constructed medical articles scented in-situ; and also is a method and system for scenting in-situ a sealed dispenser of constructed medical apparel, such as facemasks.

A number of unexpected befits and unique advantages are provided by the instant invention. Most notably, this method and system of scenting the raw fibrous materials is indirect and the structural fabric components comprising a non-woven fabric article, a woven fabric article or other fabric medical articles do not change; and are not added to or subtracted from its intended barrier performance, as defined and regulated by the FDA. Also, this method and system of delivering a fragrance of choice to a previously constructed fabric article of manufacture does not alter any material, does not modify any structure, and does not affect any physical or chemical property and feature of the fabric and medical article itself.

### I. The Medical Articles Packaged in a Container

Fabric Articles and Apparel:

There is a large range and variety of medical items comprised in whole or in part of natural and/or synthetic fabric, which are commonly manufactured as woven or non-woven fabric articles, and then are packaged in preset quantities in a closed container or dispenser. Some of these, of necessity, are sterilized prior to use; the majority of these articles, however, are not. All that is typically required is that they be clean (albeit not sterile); be easily accessible and removable from their packaging; and be functional to achieve their intended use, purpose, or result.

Merely exemplifying this range and variety of medical articles and apparel comprised of fabric are: hospital gowns and surgical scrubs; swabs, gauzes and sponges of varying sizes, materials and constructions; head covers and beard covers; cloths, towels, and sheets of differing materials and dimensions; pillow covers, bedding and drapes; air filters of various porous characteristics; hygienic tampons; and, of course, protective clothing articles such as safety hoods and facemasks. For purposes of the present invention, all these medical articles and medical apparel collectively and cumulatively constitute a one class of products.

Nevertheless, for ease of comprehension and increased clarity of description, a single illustrative and representative example and member of this class, the fully constructed facemask, will be employed herein as the fabric article of choice. It will be expressly understood and recognized, therefore, that the detailed description presented hereinafter which is relevant and pertains to a fully constructed facemask also applies—substantively, fully, and co-extensively—to all the other fabric containing articles of manufacture constituting the membership of this medical article class of products.

The Conventional Facemask:

The conventionally known and used non-woven facemask is a standard ear loop or tie string article of manufacture intended to protect the nose and mouth of the wearer. Various materials are employed in fabricating a fully constructed conventional facemask. The outer-facing of a face mask is usually formed as a pulp/polyester or polypropylene spunbond; the filter portion of a facemask typically is formed of polypropylene meltblown; the inner-facing of the facemask is primarily formed of pulp/polyester; and the binding tapes of the face mask are primarily formed of polypropylene or polyester.

The facemask is intended to be manufactured using the previously existing processes commonly employed in the technical field for making such fabricated non-woven devices. The present invention presumes that the facemask is fully constructed in all structural respects; and further presumes that whatever the particular features and properties of the individual facemask may be, that it is completely consistent with its conventionally made and previously sold predecessor versions.

It will be appreciated that every fibrous material employed in the making of a fabricated non-woven face mask has some intrinsic level of odor; and that this odor is a complex and variable smell that is the combined result of the particular fiber used as a raw material, the specific manufacturing process, the individual packaging, and the assembly plant where the manufacturing activity occurs. Also, the different fibrous materials used in combination and provided by a variety of different suppliers to make facemasks often markedly change the smell and intensity of the facemask's odor. Thus, there is no effective way to predict or even guess what the odor of an individual facemask might be; and the quality and intensity of the odor will often vary substantially from package-to-package and batch-to-batch.

### II. The Method And System For Delivering A Fragrance In-Situ

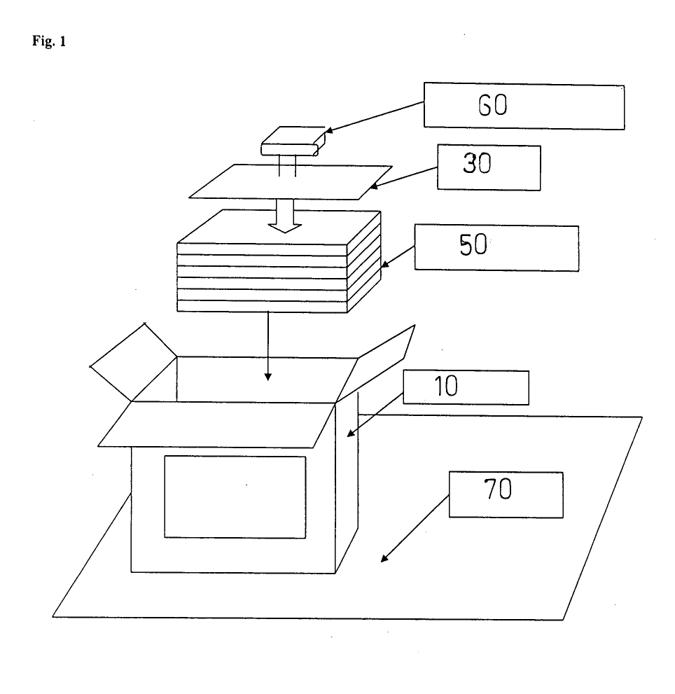

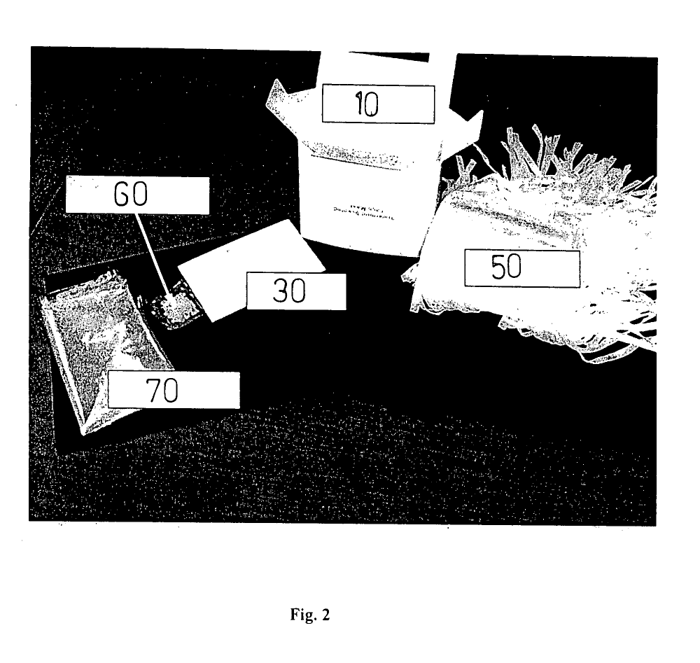

The instant invention comprises a sealed container of constructed facemask which has become scented in-situ. The system by which this result is obtained is illustrated by FIGS. 1 and 2 respectively, and is as follows:

The Dispenser Housing:

The packaging comprises a dispenser housing having at least one wall, predetermined external dimensions and configuration, and an internal volume of air. The dispenser housing is typically termed a “packer box” within the industry; often has four side walls and foldable top and bottom flaps; and can be completely closed on-demand after the facemasks have been deposited internally. Clearly the length, width and girth of the packer box can vary substantially depending upon the number of facemask it is to hold; and although the typical shape for the packer box is as a rectangular form, there is no requirement or demand at all that the overall geometric configuration for the dispenser be either geometric, symmetrical, or regular as such.

A typical rectangular-shaped packer box 10 is shown by FIGS. 1 and 2 respectively. Its sidewalls 12 and foldable top flaps 14 and bottom flaps 16 provide a fixed internal spatial volume 18 for the placement and positioning of the facemasks 50. It is preferred that the packer box 10 be constructed from clay coated board; and the internal spatial volume 18 of this housing be sufficient to hold approximately fifty individual facemasks 50, each of which is intended to be dispensed singly one at a time. Also, a sealed opening (not shown) is present in a wall of the dispenser housing which can be unsealed at will for the on-demand dispensation of a single constructed facemask at a time.

It is preferred and expected that the facemasks 50 will be pre-arranged in a stack, as is the conventional practice today in the industry, before being placed into the internal spatial volume 18 of the packer box 10. The stacking arrangement for the facemask allows them to be dispensed singly for the user's convenience.

The Air Permeable Separator Barrier:

After the facemask 50 have been placed into the internal spatial volume 18 of the dispenser housing 10, at least one permeable separator barrier 30 is then placed over the pre-arranged stack of facemasks already positioned within the packer box. As seen in FIGS. 1 and 2, the separator barrier 30 typically is a single sized sheet or divider of determinable dimensions and configuration; is routinely formed and comprised of durable paper or pulp (or of a similar solid matter); and is air permeable—i.e., will allow air to pass passively through it. The essential and requisite feature of air permeability is typically provided via the presence of at least one perforation 32 (or aperture), and preferably by a plurality of perforations 32 (or apertures), which extend over and through the material substance and thickness of the separator barrier 30.

The separator barrier 30 is placed over the arranged stack of facemasks 50 lying within the packer box 10; and the separator barrier 30 is then positioned within the inner dimensions and the internal spatial volume of the housing such that at least first and second isolated spatial pockets of air are formed internally. Thus, after the separator barrier has been placed in its intended position, a first isolated spatial pocket is structurally formed on one side of the separator barrier; and a second discrete isolated spatial pocket of air is concurrently formed on the other side of the separator barrier 30.

Both the first and the second isolated spatial pockets of air created by the placement of the separator barrier 30 serve as discrete internal containment zones within the packer box 10. Within the first isolated spatial pocket and containment zone will lie the arranged stack of facemask 50. Within the second isolated spatial pocket and containment zone will lie a prepared perfume sachet 60, which will over time provide and passively deliver a desired fragrance to the facemasks 50.

It will be noted and appreciated, therefore, that the primary characteristics of the separator barrier 30 within the packer box 10 are two: Not only does the separator barrier function as a physical divider or substantive partition; but also it has a demonstrable capability for allowing the ambient air and gases present on one side and lying within one containment zone created by the separator barrier to pass (passively by diffusion) through its perforations (or other apertures) and enter the other containment zone, and to mix with the ambient air and contents of the other containment zone within the packer box—even as the separator barrier continues to act as a physical restraint against any passage of solid matter from one isolated spatial pocket to the other.

The Perfume Sachet:

A prepared sachet (or packet) 60 of perfumed pellets 62 is then subsequently placed within the second internal spatial pocket and containment zone created by the placement of the air permeable separator barrier. It will be recognized and appreciated that the second spatial pocket and containment zone of the permeable separator barrier is a volumetric space which is sized to hold the perfumed sachet 60 and will prevent the perfumed sachet from being removed from the packer box, especially during the act of dispensing one or more facemasks on-demand.

Preferably, the sachet 60 is formed of approximately 1 mil thick polypropylene as an open walled packet; and will contain about 3 grams of intensely scented pellets as the perfume or fragrance source. Each perfumed pellet in the sachet is approximately 1/16″ in diameter and is about 0.1 grams in weight (mass). The particular fragrance or scent released by the perfumed pellets is chosen in advance of use; and typically will provide a pleasing, non-offensive aroma.

A broad range and variety of choices are available for the scents and fragrances which can be released by the pellets in the sachet. The preferred fragrance is spearmint. Among the desirable alternative choices are the scents of peppermint, cinnamon, cloves, strawberry, orange, lemon, raspberry and spicy apple.

Accordingly, when the scented pellet sachet 60 is placed into the second internal spatial pocket provided by the air permeable separator barrier, the bottom flaps of the packer box are folded into position and the disperser housing is closed. Within this closed dispenser housing, the scented pellets of the sachet 60 will continuously release a concentrated charge (dose) of fragrance into the closed ambient air constituting the environment of the second spatial pocket and containment zone. The concentration and intensity of perfume fragrance released into the closed ambient air of the second containment zone will increase over time; and such a closed environment and its ambient air with its ever-increasing intensity of fragrance will passively and ever-more quickly enter and pass through the perforations (or other apertures) of the separator barrier; and then enter the closed environment of the first isolated spatial pocket and containment zone in which the arranged stack of facemasks reside. In this manner, and by this passive diffusion mechanism of action, the concentrated charge of perfume fragrance in the ambient air of the second isolated spatial pocket and containment zone is built up over time; and is transferred to and becomes intermixed with the internal ambient air and closed environment of the first isolated spatial pocket and containment zone for contact with and impregnation of the arranged stack of facemasks.

Sealing the Dispenser Housing:

After the prepared sachet 60 of perfumed pellets has been placed into the second internal spatial pocket and containment zone created by the air permeable separator barrier 30, the bottom flaps 16 of the packer box are closed. Then, the closed packer box will be stretch wrapped with a non-porous shrink wrap film 70. Subsequently, the wrapped film 70 is heated in the conventional manner, and the closed packer box becomes sealed air-tight.

Once sealed, the dispenser housing and its internal contents become isolated and segregated from the ambient air environment. The sealed sachet of perfumed pellets and their scent is continuously released within the interior of the closed packer box with minimum leakage.

On average, it will take approximately 3 days (72 hours) time for the fragrance released by the perfumed pellets to saturate the internal air environment of the sealed packer box and to permeate the non-woven fabric substance of the facemasks contained within the packer box. It is estimated that the useful shelf life of a shrink wrapped sealed packer box is about 1 year's time. Once the sealed packer box is opened and made accessible to the open air environment, the fragrance of the scent already permeated into the substance of each facemask then lasts for about 3 weeks' time.

The present invention is not to be limited in form nor restricted in scope except by the claims appended hereto:

What we claim is:

1. A sealed container of constructed medical articles scented in-situ comprising:
a closed dispenser housing comprising at least one wall of predetermined external dimensions and configuration and having a closed internal volume of air;
a sealed opening in said wall of said dispenser housing which can be unsealed at will for the on-demand dispensation of a constructed medical article scented in-situ;
an air permeable barrier of determinable dimensions and configuration positioned within said closed internal volume of said dispenser housing such that at least first and second isolated spatial pockets of air are formed;
a predetermined number of constructed medical articles positioned within said first isolated spatial air pocket;
a fixed quantity of at least one scent-releasing fragrant material positioned within said second isolated spatial air pocket, wherein the scent of said fragrant material is released into and becomes carried by the air of said second isolated spatial pocket;
a migratory scented air mass generated within said second isolated spatial pocket which passes through said air permeable barrier and intermixes with the air of said first isolated spatial pocket, whereby said constructed medical articles become exposed to said scented air mass and become impregnated with said scent in-situ while contained within said first isolated spatial pocket of said closed dispenser housing.

2. The sealed container of constructed medical articles scented in-situ as recited in claim 1 wherein said medical article comprises at least one non-woven fabric.

3. The sealed container of constructed medical articles scented in-situ as recited in claim 1 wherein said medical article comprises at least one woven fabric.

4. The sealed container of constructed medical articles scented in-situ as recited in claim 1 wherein said medical article is apparel.

5. The sealed container of constructed medical articles scented in-situ as recited in claim 1 wherein said medical article is selected from the group consisting of hospital gowns and surgical scrubs; swabs, gauzes and sponges; head covers and beard covers; cloths, towels, and sheets; pillow covers, bedding and drapes; air filters; hygienic tampons; safety hoods;
and facemasks.

6. A sealed container of constructed facemasks scented in-situ comprising:
a closed dispenser housing comprising at least one wall of predetermined external dimensions and configuration and having a closed internal volume of air;
a sealed opening in said wall of said dispenser housing which can be unsealed at will for the on-demand dispensation of a constructed facemask scented in-situ;
an air permeable barrier of determinable dimensions and configuration positioned within said closed internal volume of said dispenser housing such that at least first and second isolated spatial pockets of air are formed;
a predetermined number of constructed facemasks positioned within said first isolated spatial air pocket;
a fixed quantity of at least one scent-releasing fragrant material positioned within said second isolated spatial air pocket, wherein the scent of said fragrant material is released into and becomes carried by the air of said second isolated spatial pocket;
a migratory scented air mass generated within said second isolated spatial pocket which passes through said air permeable barrier and intermixes with the air of said first isolated spatial pocket, whereby said constructed facemasks become exposed to said scented air mass and become impregnated with said scent in-situ while contained within said first isolated spatial pocket of said closed dispenser housing.

7. A method for making a sealed container of constructed medical articles scented in-situ, said method comprising the steps of:
obtaining a closed dispenser housing comprising at least one wall of predetermined external dimensions and configuration and having a closed internal volume of air;
introducing a sealed opening in said wall of said dispenser housing which can be unsealed at will for the on-demand dispensation of a constructed medical article scented in-situ;
positioning an air permeable barrier of determinable dimensions and configuration within said closed internal volume of said dispenser housing such that at least first and second isolated spatial pockets of air are formed;
placing a predetermined number of constructed medical articles within said first isolated spatial air pocket;
putting a fixed quantity of at least one scent-releasing fragrant material within said second isolated spatial air pocket, wherein the scent of said fragrant material is released into and becomes carried by the air of said second isolated spatial pocket as a scented air mass;
allowing said scented air mass generated within said second isolated spatial pocket to pass through said air permeable barrier and intermix with the air of said first isolated spatial pocket, whereby said constructed medical articles become exposed to said scented air mass and become impregnated with said scent in-situ while contained within said first isolated spatial pocket of said closed dispenser housing.

8. The method for making a sealed container of constructed medical articles scented in-situ as recited in claim 7 wherein said medical article comprises at least one non-woven fabric.

9. The method for making a sealed container of constructed medical articles scented in-situ as recited in claim 7 wherein said medical article comprises at least one woven fabric.

10. The method for making a sealed container of constructed medical articles scented in-situ as recited in claim 7 wherein said medical article is apparel.

11. The method for making a sealed container of constructed medical articles scented in-situ as recited in claim 7 wherein said medical article is selected from the group consisting of hospital gowns and surgical scrubs; swabs, gauzes and sponges; head covers and beard covers; cloths, towels, and sheets; pillow covers, bedding and drapes; air filters; hygienic tampons; safety hoods; and facemasks.

12. A method for making a sealed container of constructed facemasks scented in-situ, said method comprising the steps of:
obtaining a closed dispenser housing comprising at least one wall of predetermined external dimensions and configuration and having a closed internal volume of air;
introducing a sealed opening in said wall of said dispenser housing which can be unsealed at will for the on-demand dispensation of a constructed facemask scented in-situ;
positioning an air permeable barrier of determinable dimensions and configuration within said closed internal volume of said dispenser housing such that at least first and second isolated spatial pockets of air are formed;
placing a predetermined number of constructed face masks within said first isolated spatial air pocket;
putting a fixed quantity of at least one scent-releasing fragrant material within said second isolated spatial air pocket, wherein the scent of said fragrant material is released into and becomes carried by the air of said second isolated spatial pocket as a scented air mass;
allowing said scented air mass generated within said second isolated spatial pocket to pass through said air permeable barrier and intermix with the air of said first isolated spatial pocket, whereby said constructed facemasks become exposed to said scented air mass and become impregnated with said scent in-situ while contained within said first isolated spatial pocket of said closed dispenser housing.
